(19) United States
(12) Patent Application Publication
(10) Pub. No.: US 2023/0218211 A1
(43) Pub. Date: Jul. 13, 2023

(54) METHOD FOR DETERMINING AN ACTUAL CONCENTRATION OF A SUBSTRATE USING AN ARRAY OF SELF-CALIBRATED BIOSENSORS AND DEVICE FOR IMPLEMENTING THE METHOD

(71) Applicants: UNIVERSITE GRENOBLE ALPES, Saint Martin d'Hères (FR); CENTRE NATIONAL DE LA RECHERCHE SCIENTIFIQUE, Paris (FR); INSTITUT POLYTECHNIQUE DE GRENOBLE, Grenoble (FR); CENTRE HOSPITALIER UNIVERSITAIRE DE GRENOBLE ALPES, La Tronche (FR)

(72) Inventors: Abdelkader ZEBDA, Grenoble (FR); Philippe CINQUIN, Saint-Nazaire-Les-Eymes (FR); Don MARTIN, Gieres (FR)

(73) Assignees: UNIVERSITE GRENOBLE ALPES, Saint Martin d'Hères (FR); CENTRE NATIONAL DE LA RECHERCHE SCIENTIFIQUE, Paris (FR); INSTITUT POLYTECHNIQUE DE GRENOBLE, Grenoble (FR); CENTRE HOSPITALIER UNIVERSITAIRE DE GRENOBLE ALPES, La Tronche (FR)

(21) Appl. No.: 18/009,775

(22) PCT Filed: Jun. 11, 2021
(86) PCT No.: PCT/IB2021/055158

§ 371 Date: Dec. 12, 2022

(30) Foreign Application Priority Data
Jun. 12, 2020 (FR) 2006175

(51) Int. Cl. A61B 5/1495; A61B 5/145; A61B 5/1486

(52) U.S. Cl. CPC A61B 5/1495; A61B 5/14532; A61B 5/14546; A61B 5/14865; A61B 2562/046; A61B 2562/0215

(57) ABSTRACT

A method for determining a region in which the actual concentration is located, in a medium, of a substrate made up of any molecule likely to undergo catalysed oxidation-reduction by a catalyst. The method includes the following steps: taking at least one group of at least two biosensors, each biosensor having a calibration curve of the signal induced by the oxidation-reduction reaction and having identical initial portions of their calibration curves up to a concentration value of the substrate from which the measurement of the signal differ; and when more than one group is present, the biosensors in different groups having different calibration curves without identical initial portions; placing the biosensors in contact with the medium; measuring the signal induced by the oxidation or reduction reaction for each biosensor in the group/groups; comparing all the signal values produced by the biosensors and following the method described in the description.

The present invention relates to a method for determining, in a stable manner over the course of time, a region in which the actual concentration is located, in a medium, of a substrate that is likely to undergo an oxidation-reduction reaction with an enzyme, using an array of biosensors and to a device for implementing the method.

Diabetes is a global epidemic disease that affected 422 million people in 2014, compared to 108 million in 1980, and is expected to be the seventh leading cause of death worldwide if current morbidity trends continue [1,2,3]. In 2016, nearly 12% of the French population was diagnosed with diabetes [4]. In addition to increasing the mortality rate, diabetes and its complications inflict considerable economic losses and significant social security expenses (work stoppage, treatments, hospital care . . . ) [4]. Indeed, in France, the cost of diabetes treatment is about 10 billion euros, 80% of which is related to the treatment of complications. These complications could be anticipated and/or avoided with reliable real-time blood glucose measurement devices [5].

The emergence of glucose sensors has enabled patients to manage their insulin levels and thus help limit diabetes mortality. Traditional glucose testing devices include glucose sensors based on electrochemical methods (glucose meter). A glucose biosensor consists of a working electrode based on conductive materials associated with an enzyme capable of catalyzing glucose oxidation such as glucose oxidase (GOx) and glucose dehydrogenase (GDH) and a counter-electrode which can be of platinum, gold or carbon. The enzyme is immobilized on or near the surface of the electrode. Frequently, the enzyme is associated with a redox mediator which allows the transfer of electrons between the enzyme and the electrode. The most commonly used mediators in the case of GOx are ferrocene, ferrocyanide and osmium complexes. The working electrode and the counter-electrode are placed in contact with the test sample and when a voltage is applied between the working electrode and the counter-electrode an electric current flows in the circuit created by the electrodes and the test sample. This current is induced by the oxidation reaction of glucose at the working electrode which is catalyzed by the enzyme and the value of this current depends on the concentration of glucose in the test sample.

The most common blood glucose testing involves performing a number of tests each day or week by analyzing a small sample of blood [5,6], which is inconvenient, uncomfortable and therefore leads to poor patient acceptability. Furthermore, such tests do not take into account rest periods and lead to approximations of measurements. Furthermore, this type of monitoring does not provide real-time information and therefore cannot prevent hypoglycemic (<3.0 mM) and hyperglycemic (>11.1 mM) events in advance.

Continuous autonomous blood glucose monitoring is crucial to understanding the trends, direction and frequency of blood glucose changes. Interstitial glucose meters are available that allow diabetics to monitor their blood glucose in real time with patches placed on the skin. However, these devices must be replaced every two weeks because of biosensor drift. Indeed, the drift in time of enzymatic biosensors in general and glucose biosensors in particular is the major lock for the development of enzymatic biosensors able to keep their sensitivity over long periods of time without needing to be re-calibrated or replaced.

An electrochemical enzyme biosensor degrades over time. This is due to the decreasing stability of the enzymes, the decrease of their catalytic activities and the degradation of the electrode.

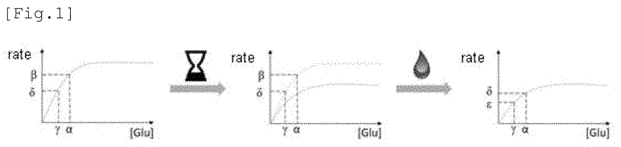

FIG. 1 shows this phenomenon and its direct impact on glucose quantification

In FIG. 1 are shown three graphs illustrating the rate of reaction occurring at the working electrode of an electrochemical biosensor as a function of glucose concentration. In the left graph, the glucose concentration at a given rate is determined (rates β and δ correspond to bloog glucose rates (BGR) of α and γ). Over time, enzymatic degradation changes the reaction rates. This can be seen in the middle graph of FIG. 1: the BGR a is no longer defined by the rate β but by the rate δ. The values delivered by the biosensor are therefore no longer reliable. Thus, a calibration is necessary, represented by the graph on the right. The calibration is performed as follows:
(1) The user measures his or her BGR via a drop of blood and transmits his or her current BGR equal to a to the biosensor.
(2) The rate δ measured by the sensor at the same instant is recorded.
(3) The program contained in the sensor associating a rate with a BGR is reset with the two measured values.

The catalytic activity is not restored. After a certain time, the biosensor will have lost too much precision and will have to be replaced.

Therefore, for any enzymatic biosensor measurement, drift is inevitable and external calibration is necessary. For this reason, the development of implantable glucose biosensors capable of operating over long periods of time is currently out of reach. Furthermore, in general, for all enzymatic biosensors this measurement drift is considered to be the major reason for their non-emergence in the market.

Current glucose biosensors designed to measure blood glucose levels are limited by biosensor drift over time, which is a major impediment to the development of implantable or non-implantable glucose biosensors capable of operating over long periods of time and continuously measuring blood glucose levels in diabetics.

Therefore, there is a need for an enzymatic glucose biosensor that is able to “self-calibrate” away from the measurement drift of existing biosensors and continuously monitor blood glucose with long-term stability without the need for replacement or external calibration. This is achieved by comparing the measured values of a set of biosensors of known characteristics and not on the basis of the values as they are measured.

The present invention thus relates to a method for determining, in a stable manner over the course of time, a region in which the actual concentration is located, in a medium, of a substrate (S₁) made up of any molecule likely to undergo catalyzed oxidation-reduction by a catalyst,

characterized by the fact that the method comprises the following steps:
a) taking at least one group of at least two biosensors, each biosensor having a calibration curve of the signal induced by the oxidation-reduction reaction:
   the biosensors in a group having identical initial portions of their calibration curves up to a concentration value of substrate (S₁), referred to as the separation concentration (SC), from which the measurement of the signal differs from one biosensor in the group to another; and
   when more than one group is present, the biosensors in different groups having different calibration curves without having identical initial portions;
b) placing the biosensors in contact with said medium;
c) measuring the signal induced by the oxidation or reduction reaction for each of the biosensors in the group or groups;
d) comparing all the signal values produced by all the biosensors, and in the case of a single group of biosensors:
   if all signal values are equal, the concentration of the substrate (S₁) is less than or equal to the lowest SC;
   if all signal values are different, the concentration of the substrate (S₁) is higher than the highest SC;
   if a part of the biosensors has the same signal values, the concentration of the substrate (S₁) is less than or equal to the lowest SC of the biosensors in that part and greater than the SC of the biosensor with the next lowest SC; and
   in the case of more than one group of biosensors:
   if all signal values in each group are equal, the concentration of the substrate (S₁) is less than or equal to the lowest SC;
   if all signal values are different in all groups, the concentration of the substrate (S₁) is higher than the highest SC;
   if a part of the biosensors in a group has the same signal values, the concentration of the substrate (S₁) is less than or equal to the lowest SC of the biosensors in that part and greater than the SC of the biosensor with the next lowest SC in the relevant group;
   if in one part of the groups of biosensors all biosensors in a group have the same signal value and in the remaining part all biosensors in a group have a different signal value, the concentration is less than or equal to the lowest SC of the group or groups with the same signal values in each group, and greater than the highest SC of the group or groups with different signal values in each group.

Since the separation concentrations are determined by successive comparison between two calibration curves, for a group with n biosensors and n corresponding calibration curves, there are n−1 separation concentrations for this group.

The signal can be an electrochemical signal, each biosensor then comprising a working electrode, a reference electrode and a counter-electrode between which a current induced by the oxidation or reduction reaction passes, the electrochemical signal being either the intensity of this current or the potential difference between the electrodes during the oxidation-reduction reaction of the substrate (S₁).

For each biosensor, the working electrode can be a carbon, gold or platinum electrode, the reference electrode can be a platinum, gold or diamond electrode, and the reference electrode can be a silver chloride electrode.

The catalyst may be an enzymatic catalyst or a chemical catalyst, the chemical catalyst being an abiotic catalyst selected in particular from platinum, platinum nanostructures, platinum alloy nanostructures, gold nanostructures and gold alloy nanostructures, or a molecular catalyst selected in particular from a porphyrin-gold complex and a porphyrin-rhodium complex.

When the catalyst is an enzymatic catalyst, a mediator may be associated with said catalyst, said mediator being notably selected from among ferrocene, ferrocyanide, osmium complexes, quinone derivatives such as naphthoquinone, and phenothiazine derivatives.

A substrate (S₁) transporter can be arranged on the biosensor or biosensors.

The biosensors in each group may differ in at least one parameter selected from:

p1: the amount of catalyst;

p2 the oxidation-reduction Km of the catalyst, in case the latter is an enzymatic catalyst or the saturation limit of the catalyst in case the latter is a chemical catalyst;

p3: the amount of a mediator of the catalyst, if present, in case the catalyst is an enzymatic catalyst; or

p4: the transport Km of a substrate (S₁) transporter if present.

The oxidation-reduction Km of the enzymatic catalyst is the Michaelis constant of the catalyst. It represents the substrate concentration for which the reaction rate is half of the maximum rate.

The Michaelis constant of an enzymatic catalyst is specific to that catalyst and depends on the organism from which the catalyst is derived and its extraction process.

For example, for commercial glucose oxidase enzymes, the Michaelis constant is 1.34 mM for a glucose oxidase from P. ostreatus, 5.7 mM for a glucose oxidase from P. amagasakiense, 6.2 mM for a glucose oxidase from P. pinophilum, 10.2 mM for a glucose oxidase from T. flavus, 30 for a glucose oxidase from A. niger and more than 38 mM for a glucose oxidase from P. chrysosporium.

The saturation limit of the chemical catalyst is the concentration of the substrate (S₁) at which the measurement of the signal induced by the oxidation-reduction reaction of the substrate (S₁) reaches a maximum limit value. Km is the substrate concentration required for the signal induced by the oxidation-reduction reaction of the substrate to reach half the saturation limit. The saturation limit can be varied because it depends on the nature of the chemical catalyst used.

The transport Km of a substrate transporter is the Michaelis constant of the transporter. It represents the substrate concentration for which the transport rate of the substrate is half of the maximum rate.

As an example, the transport Km of the glucose transporter Glu1 is 3 mM, Glu2 is 17 mM, Glu3 is 1.8 mM, Glu4 is 5 mM, Glu8 is 2.4 mM and Glu9 is 0.5 mM.

Biosensors (BC) can be noted as follows:

BC₁₁ . . . BC₁ᵢ . . . BC₁ₙ . . .

BC₂₁ . . . BC₂ᵢ . . . BC₂ₙ . . .

BCⱼ₁ . . . BCⱼᵢ . . . BCⱼₙ . . .

BCₘ₁ . . . BCₘᵢ . . . BCₘₙ . . .

m and n each being an integer, m being the number of groups and n being the number of biosensors in each group, the biosensors BC₁₁ to BC₁ₙ belonging to group 1, the biosensors BCₘ₁ to BCₘₙ belonging to group m,

between each biosensor in a group, a parameter selected from p1 to p4 is varied; and between the biosensors of two different groups, another of these parameters p1 to p4 is varied.

As an example, the amount of catalyst mediator is varied between each of the biosensors in a first group so that each biosensor in that group has a different calibration curve.

Then, in order to obtain a second different group of biosensors, the same variation of the amount of mediator of the catalyst can be kept between each of the biosensors in this second group, and to create a difference between the first and second groups, a second parameter is varied between these two groups, for example the oxidation-reduction Km of the catalyst. The Km could for example be chosen at a first value for each of the biosensors in the first group and be chosen at a second value, different from the first, for each of the biosensors in the second group.

In particular:
the substrate (S₁) can be glucose or lactate;
the catalyst may be an enzymatic catalyst selected from glucose oxidase, glucose dehydrogenase and cellobiose dehydrogenase, or lactate oxidase or lactate dehydrogenase;
the enzymatic catalyst mediator, if present, may be selected from ferrocene, ferrocyanide, osmium complexes, quinone derivatives such as naphthoquinone, and phenothiazine derivatives;
the substrate (S₁) transporter, if present, can be, if necessary, a glucose transporter which is chosen in particular from GLUT1, GLUT2, GLUT3, GLUT4, GLUT6, GLUT8, GLUT10 and GLUT12, or can be a lactate transporter which is chosen in particular from MCT1, MCT2, MCT3, MCT4.

The present invention also relates to a device for implementing the method as defined above, characterized by the fact that it comprises at least one group of at least two biosensors, each biosensor having a calibration curve for the signal induced by the oxidation-reduction reaction:
the biosensors in a group having identical initial portions of their calibration curves up to a concentration value of the substrate (S₁) referred to as the separation concentration (SC) from which the measurement of the signal differs from one biosensor in the group to another; and
when more than one group is present, the biosensors in different groups having different calibration curves without having identical initial portions between the groups, each biosensor being able to measure a signal induced by a catalytic oxidation-reduction reaction of the substrate (S₁) and

each biosensor comprising:
a catalyst;
in the case of an enzymatic catalyst, if applicable, a mediator thereof; and
if applicable a substrate (S₁) transporter.

Each biosensor can be capable of measuring an electrochemical signal and then comprises a working electrode, a reference electrode and a counter-electrode between which passes a current induced by the oxidation or reduction reaction of the substrate (S₁), the electrochemical signal being either the intensity of this current or the potential difference between the electrodes during the oxidation-reduction reaction of the substrate (S₁).

For each biosensor, the working electrode can be a carbon, gold or platinum electrode, the reference electrode can be a platinum, gold or diamond electrode, and the reference electrode can be a silver chloride electrode.

The catalyst may be an enzymatic catalyst or a chemical catalyst, it being possible for the chemical catalyst to be an abiotic catalyst chosen in particular from platinum, platinum nanostructures, platinum alloy nanostructures, gold nanostructures and gold alloy nanostructures, or to be a molecular catalyst chosen in particular from a porphyrin-gold complex and a porphyrin-rhodium complex.

When the catalyst is an enzymatic catalyst, a mediator may be associated with said catalyst, said mediator being notably selected from among ferrocene, ferrocyanide, osmium complexes, quinone derivatives such as naphthoquinone, and phenothiazine derivatives.

The biosensors in each group may differ in at least one parameter selected from:

p1: the amount of catalyst;

p2 the oxidation-reduction Km of the catalyst, in case the latter is an enzymatic catalyst or the saturation limit of the catalyst in case the latter is a chemical catalyst;

p3: the amount of a mediator of the catalyst, if present, in case the catalyst is an enzymatic catalyst; or

p4: the transport Km of a substrate (S₁) transporter if present.

Biosensors (BC) can be noted:

BC₁₁ . . . BC₁ᵢ . . . BC₁ₙ . . .

BC₂₁ . . . BC₂ᵢ . . . BC₂ₙ . . .

BCⱼ₁ . . . BCⱼᵢ . . . BCⱼₙ . . .

BCₘ₁ . . . BCₘᵢ . . . BCₘₙ . . .

m and n each being an integer, m being the number of groups and n being the number of biosensors in each group, the biosensors BC₁₁ to BC₁ₙ belonging to group 1, the biosensors BCₘ₁ to BCₘₙ belonging to group m,

between each biosensor in a group, a parameter selected from p1 to p4 is varied; and between the biosensors of two different groups, another of these parameters p1 to p4 is varied.

In particular:
the substrate (S₁) can be glucose or lactate;
the catalyst may be an enzymatic catalyst selected from glucose oxidase, glucose dehydrogenase and cellobiose dehydrogenase, or lactate oxidase or lactate dehydrogenase;
the enzymatic catalyst mediator, if present, may be selected from ferrocene, ferrocyanide, osmium complexes, quinone derivatives such as naphthoquinone, and phenothiazine derivatives;
the substrate (S₁) transporter, if present, can be, if necessary, a glucose transporter which is chosen in particular from GLUT1, GLUT2, GLUT3, GLUT4, GLUT6, GLUT8, GLUT10 and GLUT12, or can be a lactate transporter which is chosen in particular from MCT1, MCT2, MCT3, MCT4.

In a particular embodiment, the biosensors may be arranged on a support, the working electrode, the reference electrode and the counter-electrode being screen-printed on said support.

The support on which the biosensors are arranged can be selected from glass plates, plastic plates, such as polyethylene terephthalate plates, ceramic plates, such as alumina or a composite between alumina and another ceramic, nylon plates, silicon plates, polystyrene-based films, or polyester sheets.

The catalyst can be deposited on the working electrode of each biosensor by encapsulation, grafting, absorption or trapping.

When the catalyst is an enzymatic catalyst, the catalyst may be present on the surface of the working electrode of each biosensor by being applied on said surface of the working electrode inside a protective layer, the protective layer being in particular, of chitosan, Nafion, polypyrrole, or polyacrylic acid, or of a conductive polymer such as polyaniline, polylactic acid, polydopamine, or polyethylene glycol. The enzymatic catalyst mediator may be enclosed with said enzymatic catalyst within the protective layer.

A substrate (S₁) transporter can be present by being applied as a layer to the working electrode or optionally on the protective layer comprising the enzymatic catalyst, its mediator optionally being present, by depositing a layer of proteoliposomes enclosing the substrate (S₁) transporter.

The support on which the biosensors are arranged and the biosensors can be coated with a layer of chitosan, poly(2-hydroxyethyl methacrylate), poly(4-vinylpyridine-co-styrene) or alumina.

The device according to the invention can be arranged on the skin of a user or can be implanted in the user in order to determine a region in which the actual concentration of the substrate (S₁) in a medium is located, said substrate being glucose and said medium being blood.

In particular, for an implantable device for the determination of glucose concentration, biosensors may be chosen such that they allow the determination of the actual glucose concentration in a concentration range between 2 and 10 mM with an accuracy of 0.5 to 1 mM. That is, biosensors with separation concentrations of 2 to 10 mM with a step size of 0.5 or 1 mM should be chosen.

The following examples illustrate the present invention without limiting its scope.

### EXAMPLE 1: SELF-CALIBRATED BIOSENSOR BASED ON THE VARIATION OF THE AMOUNT OF MEDIATOR [M] AND THE AMOUNT OF ENZYME [E]

An array of 6 pairs of biosensors is fabricated on the surface of a glass plate. Electrodes are printed by screen-printing on the glass plate. This method of deposition consists in printing on a solid support a carbon electrode from a carbon ink using a printing device. Each biosensor is composed of a carbon working electrode, a platinum counter-electrode and an Ag/AgCl reference electrode.

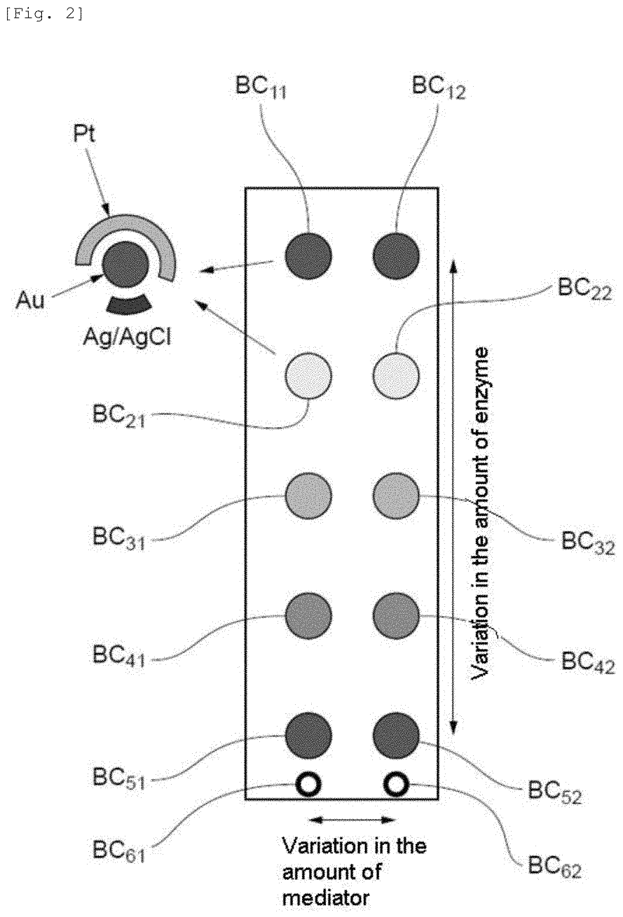

FIG. 2 shows the arrangement of the working electrode, counter-electrode and reference electrode for each biosensor. The working electrode of each biosensor is fabricated by screen-printing from a carbon ink containing 40 wt % of carbon powder dispersed in an organic solution composed of terpineol and ethyl cellulose. The reference electrodes and counter electrodes of each biosensor are made by screen printing respectively from a platinum ink containing 40 wt % of platinum powder dispersed in an organic solution composed of terpineol and ethyl cellulose and a silver ink containing 40% by weight of silver powder dispersed in an organic solution composed of terpineol and ethyl cellulose.

On each working electrode, 10 μL of a 5% volume solution of nafion-tetrabutylammonium bromide (TBAB) containing the enzyme glucose oxidase (GOx) and its mediator naphthoquinone in the amounts shown in Table 1 below is applied. This is allowed to air dry at room temperature for six hours.

This creates an array of twelve biosensors belonging to six groups of two biosensors. The biosensors within the first group are noted BC₁₁ and BC₁₂, the biosensors within the second group BC₂₁ and BC₂₂, and BC₃₁ and BC₃₂ for the third, BC₄₁ and BC₄₂ for the fourth, BC₅₁ and BC₅₂ for the fifth and BC₆₁ and BC₆₂ for the sixth.

Within each group of two biosensors, the biosensors have the same amount of GOx enzyme and show a different amount of mediator. From one group to another, the amounts of GOx enzyme and mediator change.

The biosensors are then calibrated. This initial calibration consists in measuring the intensity of the current induced by an oxidation-reduction reaction for glucose standard solutions with a known glucose concentration. Glucose is oxidized at the working electrode while oxygen is reduced at the counter electrode.

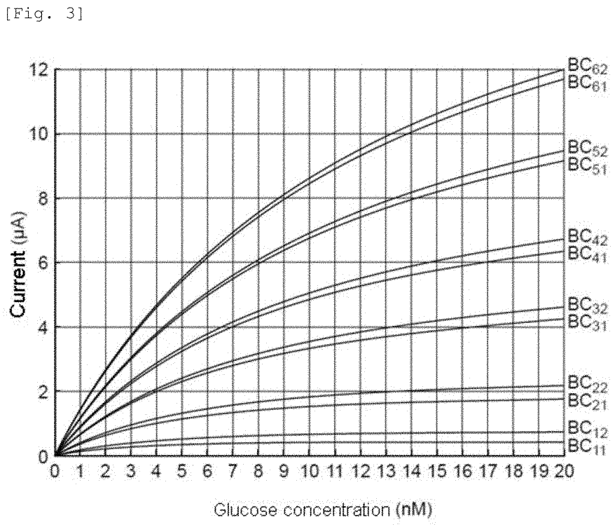

FIG. 3 shows the calibration curves that were determined for each of the biosensors of the six groups. These curves make it possible to obtain the separation concentrations from which the current intensity of the oxidation-reduction reaction of glucose becomes different between the two biosensors of the same group.

Table 1 also shows the separation concentration (SC) values found.

TABLE 1

| | | Glucose oxidase | Naphthoquinone |
|---|---|---|---|
| | Separation | concentration | concentration |
| Biosensors | concentration | [E]. | [M]. |
| BC₁₁ | 1 mM | 0.02 mM | 7 mM |
| BC₁₂ | | | 10 mM |
| BC₂₁ | 2 mM | 0.01 mM | 11 mM |
| BC₂₂ | | | 13 mM |
| BC₃₁ | 3 mM | 0.007 mM | 13 mM |
| BC₃₂ | | | 15 mM |
| BC₄₁ | 4 mM | 0.005 mM | 17 mM |
| BC₄₂ | | | 19 mM |
| BC₅₁ | 5 mM | 0.004 mM | 20 mM |
| BC₅₂ | | | 22 mM |
| BC₆₁ | 6 mM | 0.003 mM | 22 mM |
| BC₆₂ | | | 24 mM |

In order to determine the glucose concentration region of a sample to be analyzed, the groups of biosensors are brought into contact with said sample and the value of the current measured on each biosensor is measured and the value of the glucose concentration is determined according to the following method.

If the values measured at the two biosensors BC₁₁ and BC₁₂ are identical as well as those of the other biosensors, it means that the glucose concentration in the sample is lower than the separation concentration of this group of biosensors BC₁₁ and BC₁₂ which is 1 mM and is the lowest separation concentration among the groups.

If the values measured at the two biosensors BC₁₁ and BC₁₂ are different, it means that the glucose concentration in the sample is higher than the separation concentration of this group of biosensors BC₁₁ and BC₁₂ which is 1 mM.

The values measured at the next group of biosensors, namely the BC₂₁ and BC₂₂ biosensors, are then compared. If the values measured at the two biosensors BC₂₁ and BC₂₂ are identical, this means that the glucose concentration in the sample is lower than the separation concentration of the biosensor group BC₂₁ and BC₂₂ which is 2 mM. By doing so, we then know that the glucose concentration in the sample is between 1 mM and 2 mM.

The same procedure is followed for the following biosensors, i.e. if the values measured at the BC₂₁ and BC₂₂ biosensors are different and the values measured at the BC₃₁ and BC₃₂ biosensors are the same, then the glucose concentration is between 2 mM and 3 mM, and so on for the following biosensor groups.

If all the values of all the biosensors are different, it means that the concentration of glucose in the sample is higher than the highest separation concentration which is the one of the last group of biosensors BC₆₁ and BC₆₂, namely 6 mM.

Thus, the method allows for the surrounding of glucose concentration by comparing the values of the intensity of the glucose oxidation-reduction reaction measured within each biosensor for each group.

This process avoids the drift of the biosensors because it is no longer the directly measured values that allow the glucose concentration to be established but the comparison between the values measured at each biosensor.

### EXAMPLE 2: SELF-CALIBRATED BIOSENSOR BASED ON THE VARIATION OF THE KM OF THE ENZYME AND THE AMOUNT OF MEDIATOR

An array of 10 groups of two biosensors is deposited on the surface of a glass plate. The working electrode, counter-electrode and reference electrode are printed as in Example 1.

On each of the working electrodes, 10 μL of a nafion-TBAB solution containing 3 mg/mL of glucose oxidase enzyme and its mediator naphthoquinone is deposited in the amounts shown in Table 2. The Km of glucose oxidase enzymes varies between biosensors and is shown in Table 2.

We thus create an array of twenty biosensors belonging to ten couples of biosensors. This network can be noted as follows:
BC_{11 . . . 12}
BC_{21 . . . 22}
BC_{31 . . . 32}
BC_{41 . . . 42}
BC_{51 . . . 52}
BC_{61 . . . 62}
BC_{71 . . . 72}
BC_{81 . . . 82}
BC_{91 . . . 92}
BC_{101 . . . 102}

Within each couple, the Km of glucose oxidase is the same and the amount of mediator is different. From one couple to another, only the Km of glucose oxidase is different.

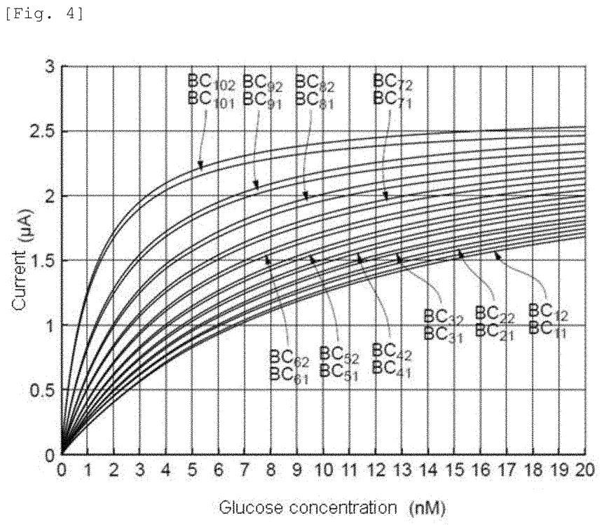

FIG. 4 shows the calibration curves that were determined for each of the biosensors of the ten groups. These curves make it possible to obtain the separation concentrations from which the current intensity of the oxidation-reduction reaction of glucose becomes different between the two biosensors of the same group.

TABLE 2

| | | | Amount of |
|---|---|---|---|
| | Separation | Km of Glucose | naphthoquinone |
| Biosensors | concentration | oxidase | (μg) |
| BC₁₁ | 4.5 mM | 5 | 18 |
| BC₁₂ | | | 20 |
| BC₂₁ | 4.3 mM | 10 | 18 |
| BC₂₂ | | | 20 |
| BC₃₁ | 3.8 mM | 15 | 18 |
| BC₃₂ | | | 20 |
| BC₄₁ | 3.5 mM | 20 | 18 |
| BC₄₂ | | | 20 |
| BC₅₁ | 3.1 mM | 25 | 18 |
| BC₅₂ | | | 20 |
| BC₆₁ | 2.7 mM | 30 | 18 |
| BC₆₂ | | | 20 |
| BC₇₁ | 2.2 mM | 35 | 18 |
| BC₇₂ | | | 20 |
| BC₈₁ | 1.8 mM | 40 | 18 |
| BC₈₂ | | | 20 |
| BC₉₁ | 1.3 mM | 45 | 18 |
| BC₉₂ | | | 20 |
| BC₁₀₁ | 1.2 mM | 50 | 18 |
| BC₁₀₂ | | | 20 |

The same procedure as in Example 1 is used to determine the glucose concentration range.

For example, if all the values of the signals of the pairs of biosensors BC₁₁, BC₁₂; BC₂₁, BC₂₂; BC₃₁, BC₃₂; BC₄₁, BC₄₂; BC₅₁, BC₅₂; are identical between each pair of biosensors, it means that the concentration of glucose is lower than the lowest separation concentration of these pairs. Here, it is the concentration of the couple BC₅₁, BC₅₂ which is 3.1 mM.

Therefore, the signal values of the other biosensors are different, which means that the glucose concentration is higher than the highest separation concentration of these couples. Here, it is the concentration of the couple BC₆₁, BC₆₂ which is 2.7 mM.

The glucose concentration is therefore in the range of 2.7 mM to 3.1 mM.

### EXAMPLE 3: BIOMIMETIC “SELF-CALIBRATED” BIOSENSOR

A group of four biosensors are deposited on the surface of a glass plate. The working electrode, counter-electrode and reference electrode are printed as in Example 1.

On each carbon working electrode 1, a layer 2 of polyaniline (PANI) from a potassium phosphate buffer solution (100 mM, pH 7) containing 2 mM aniline and 2 mg/mL glucose oxidase (GOx) enzyme 3 is deposited by electropolymerization.

Electropolymerization is performed by cyclic voltammetry between −0.5 V and 1 V (5 cycles, scan rate 10 mV/s) in a three-electrode cell with a platinum wire as counter electrode and a silver wire as reference electrode.

On each working electrode on which a polyaniline layer has been deposited, 100 μL of a potassium phosphate buffer solution (100 mM, pH 7) containing proteoliposomes containing glucose transporter proteins 5 with a Km of 0.3 mM on the first carbon electrode, 0.6 mM on the second carbon electrode, 2 mM on the third carbon electrode, and 5 mM on the fourth carbon electrode are applied. The concentration of the phosphate buffer solution in glucose transporter is 0.11 mg/mL. The diameter of the proteoliposomes 100 nm to 200 nm. We wait for 20 minutes to allow the fusion of proteoliposomes 4 to the surface of each of the working electrodes.

The fusion of the proteoliposomes 4 allows the formation of a flat lipid double layer 6 on the surface of the working electrode in which the glucose transporter 5 is located.

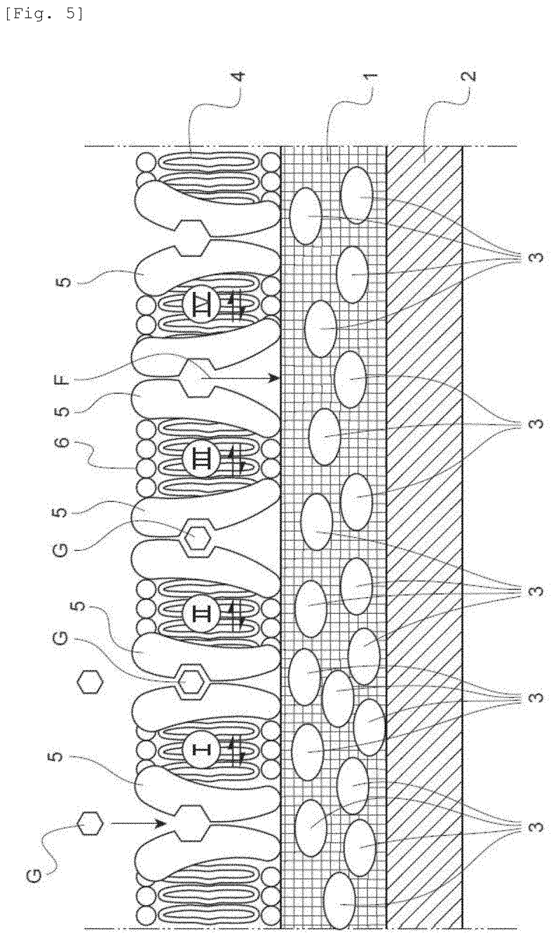

FIG. 5 shows a working electrode 1 of a biosensor and the approach of glucose G to the surface of the working electrode 1. In a first step I, glucose G approaches the glucose transporter protein 5. In a step II, glucose G is taken up by the glucose transporter protein 5. In a step III, glucose G is released from glucose transporter protein and travels to polyaniline layer 2 in which the GOx enzyme 3 is located in the direction of arrow F where glucose G will be oxidized. In a step IV, the glucose transporter protein 5 is again ready to receive glucose. The glucose transporter protein modulates the access of glucose to the glucose oxidase (Gox) deposited on the surface of the working electrode.

By calibration, the separation concentration between the biosensors can be determined. In this case, the calibration curves are presented in the form of histograms for each of the biosensors. In this case, the separation concentration corresponds to the concentration at which the current value reaches a plateau for a biosensor, as the glucose transporter can no longer transport more glucose to the working electrode.

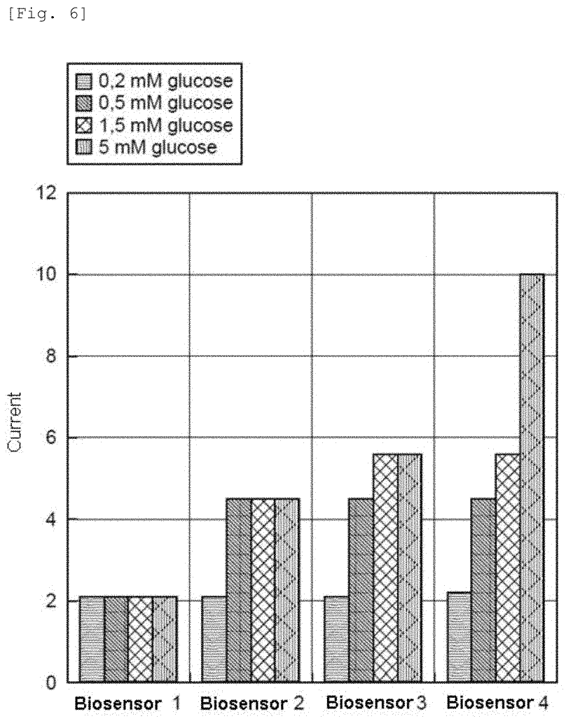

FIG. 6 shows the calibration curves of the four biosensors in Example 3.

Table 3 shows the Km of the glucose transporter and the separation concentrations between the biosensors.

TABLE 3

| | | |
|---|---|---|
| | Km of the glucose | Separation |
| Biosensor | transporter | concentration |

| | | | |
|---|---|---|---|
| 1 | 0.3 | mM | 0.2 mM |
| 2 | 0.6 | mM | 0.5 mM |
| 3 | 2 | mM | 1.5 mM |
| 4 | 5 | mM | — |

In order to determine the glucose concentration region of a sample to be analyzed, the four biosensors are brought into contact with said sample and the value of the current measured on each biosensor is measured and the value of the glucose concentration is determined by the following method.

If all values measured at each of the biosensors are equal, it means that the glucose concentration is less than or equal to the lowest separation concentration of 0.2 mM.

If the value measured for the first biosensor is different from those of the other biosensors and the values for each of the other three biosensors are the same, then the glucose concentration is greater than the separation concentration of the first biosensor and less than or equal to the separation concentration of the second biosensor. The glucose concentration will therefore be greater than 0.2 mM and less than or equal to 0.5 mM.

If all the values measured by the biosensors are different, this means that the glucose concentration is higher than the separation concentration of the third biosensor, i.e. 1.5 mM.

### EXAMPLE 4: IMPLANTABLE BIOSENSOR

A set of 10 groups of two biosensors is deposited on the surface of a silicon wafer. Working electrode, counter-electrode and reference electrode are printed as in Example 1. The enzyme glucose oxidase and its mediator are then applied to each working electrode in the amounts shown in Example 1.

The silicon plate is then covered with a biocompatible chitosan membrane by immersion in a 2% chitosan solution at room temperature for 10 s. Then, the plate is left to dry for 8 hours at 4° C.

This results in a biosensor that can be implanted under the skin of a user, in order to be able to measure the glucose concentration in the user's blood.

### EXAMPLE 5: SELF-CALIBRATED BIOSENSOR FOR MEASURING LACTATE CONCENTRATION

Two biosensors are deposited on the surface of a glass plate. The working electrode, counter-electrode and reference electrode are printed as in Example 1.

On each working electrode, 10 μL of a 1% volume chitosan solution containing the enzyme lactate oxidase (LOx) and its mediator naphthoquinone in the amounts shown in Table 4 below is applied. This is allowed to air dry at room temperature for six hours.

TABLE 4

| | Lactate oxidase | Naphthoquinone |
|---|---|---|
| | concentration | concentration |
| Biosensors | [E] | [M] |
| A1 | 0.04 mM | 1 mM |
| A2 | | 10 mM |

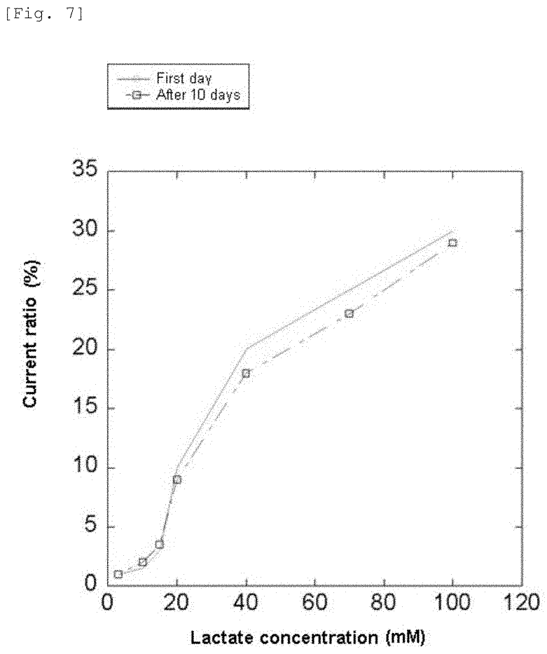

FIG. 7 shows a plot of the ratio of the current intensity measured for biosensors A1 and A2 as a function of the lactate concentration over time.

As can be seen in FIG. 7, the ratio of current intensity measured for biosensors A1 and A2 is similar on the first day of the measurement and ten days after the first measurement.

This means that the drift over time of the A1 and A2 biosensors is identical. Therefore, using a set of biosensors, it is possible to determine the lactate concentration region of a sample to be analyzed.

### REFERENCES

[1] Global report on diabetes (2016). World Health Organization
[2] International Diabetes federation (2016)
[3] Mechanisms of diabetic complications. Forbes J M1, Cooper M E. Physiol Rev. 2013 January; 93(1):137-88. doi: 10.1152/physrev.00045.2011
[4] Institut de veille sanitaire (France)
[5] Home Blood Glucose Biosensors: A Commercial Perspective. Jeffrey D. Newman & Anthony P. F. Turner. Biosensors and Bioelectronics, Volume 20, Issue 12, 20th Anniversary of Biosensors and Bioelectronics, 15 Jun. 2005, Pages 2435-2453
[6] Electrochemical Glucose Biosensors. J. Wang. Chem. Rev., 2008, 108 (2), pp 814-825

1-27. (canceled)

28. A method for determining, in a stable manner over the course of time, a region in which the actual concentration is located, in a medium, of a substrate made up of any molecule likely to undergo catalyzed oxidation-reduction by a catalyst,
wherein the method comprises the following steps:
taking at least one group of at least two biosensors, each biosensor having a calibration curve of the signal induced by the oxidation-reduction reaction:
the biosensors in a group having identical initial portions of their calibration curves up to a concentration value of the substrate, referred to as the separation concentration, from which the measurement of the signal differs from one biosensor in the group to another; and
when more than one group is present, the biosensors in different groups having different calibration curves, without having identical initial portions;
placing the biosensors in contact with the medium;
measuring the signal induced by the oxidation or reduction reaction for each of the biosensors in the group or groups;
comparing all the signal values produced by all the biosensors, and in the case of a single group of biosensors:
if all signal values are equal, the concentration of the substrate is less than or equal to the lowest separation concentration;
if all signal values are different, the concentration of the substrate is higher than the highest separation concentration;
if a part of the biosensors has the same signal values, the concentration of the substrate is less than or equal to the lowest separation concentration of the biosensors in that part and greater than the separation concentration of the biosensor with the next lowest separation concentration; and
in the case of more than one group of biosensors:
if all signal values in each group are equal, the concentration of the substrate is less than or equal to the lowest separation concentration;
if all signal values are different in all groups, the concentration of the substrate is higher than the highest separation concentration;
if a part of the biosensors in a group has the same signal values, the concentration of the substrate is less than or equal to the lowest separation concentration of the biosensors in that part and greater than the separation concentration of the biosensor with the next lowest separation concentration in the relevant group;
if in one part of the groups of biosensors all biosensors in a group have the same signal value and in the remaining part all biosensors in a group have a different signal value, the concentration is less than or equal to the lowest separation concentration of the group of groups with the same signal values in each group, and greater than the highest separation concentration of the group or groups with different signal values in each group.

29. The method according to claim 28, wherein the signal is an electrochemical signal, each biosensor then comprising a working electrode, a reference electrode and a counter-electrode between which a current induced by the oxidation or reduction reaction passes, the electrochemical signal being either the intensity of this current or the potential difference between the electrodes during the oxidation-reduction reaction of the substrate.

30. The method according to claim 29, wherein for each biosensor the working electrode is a carbon, gold or platinum electrode, the reference electrode is a platinum, gold or diamond electrode and the reference electrode is a silver chloride electrode.

31. A process according to claim 29, wherein the catalyst is an enzymatic catalyst or a chemical catalyst, it being possible for the chemical catalyst to be an abiotic catalyst chosen from platinum, platinum nanostructures, platinum alloy nanostructures, gold nanostructures and gold alloy nanostructures, or to be a molecular catalyst chosen from a porphyrin-gold complex and a porphyrin-rhodium complex.

32. The process according to claim 31, in which the catalyst is an enzymatic catalyst, wherein a mediator is associated with the enzymatic catalyst, the mediator being chosen from ferrocene, ferrocyanide, osmium complexes, quinone derivatives, and phenothiazine derivatives.

33. The method according to claim 28, wherein a substrate transporter is arranged on the biosensor or biosensors.

34. The method according to claim 28, wherein the biosensors of each group differ in at least one parameter selected from:
p1: the amount of catalyst;
p2: the Michaelis constant of the catalyst, in case the catalyst is an enzymatic catalyst or the saturation limit of the catalyst in case the catalyst is a chemical catalyst;
p3: the amount of a mediator of the catalyst, if present, in case the catalyst is an enzymatic catalyst; or
p4: the Michaelis constant of a substrate transporter if present.

35. The method according to claim 34, wherein the biosensors (BC) are noted:
BC₁₁ . . . BC₁ᵢ . . . BC₁ₙ . . .
BC₂₁ . . . BC₂ᵢ . . . BC₂ₙ . . .
BCⱼ₁ . . . BCⱼᵢ . . . BCⱼₙ . . .
BCₘ₁ . . . BCₘᵢ . . . BCₘₙ . . .
m and n each being an integer, m being the number of groups and n being the number of biosensors in each group, the biosensors BC₁₁ to BC₁ₙ belonging to group 1, the biosensors BCₘ₁ to BCₘₙ belonging to group m,
between each biosensor in a group, a parameter selected from p1 to p4 is varied; and between the biosensors of two different groups, another of these parameters p1 to p4 is varied.

36. The method according to claim 28, wherein:
the substrate is glucose;
the catalyst is an enzymatic catalyst selected from a glucose oxidase, a glucose dehydrogenase and a cellobiose dehydrogenase;
the enzymatic catalyst mediator, if present, is selected from ferrocene, ferrocyanide, osmium complexes, quinone derivatives, and phenothiazine derivatives;
the substrate transporter, if present, is a glucose transporter which is selected from GLUT1, GLUT2, GLUT3, GLUT4, GLUT6, GLUT8, GLUT10 and GLUT12.

37. The method according to claim 28, wherein:
the substrate is lactate;
the catalyst is an enzymatic catalyst selected from a lactate oxidase or a lactate dehydrogenase;
the enzymatic catalyst mediator, if present, is selected from ferrocene, ferrocyanide, osmium complexes, quinone derivatives, and phenothiazine derivatives;
the substrate transporter, if present, is a lactate transporter which is chosen from MCT1, MCT2, MCT3, MCT4.

38. A device for implementing the method according to claim 28, wherein the device comprises at least one group of at least two biosensors, each biosensor having a calibration curve for the signal induced by the oxidation-reduction reaction:
the biosensors in a group having identical initial portions of their calibration curves up to a concentration value of the substrate, referred to as the separation concentration from which the measurement of the signal differs from one biosensor in the group to another; and
when more than one group is present, the biosensors in different groups having different calibration curves without having identical initial portions between the groups, each biosensor being able to measure a signal induced by a catalytic oxidation-reduction reaction of the substrate and
each biosensor comprising:
a catalyst;
in the case of an enzymatic catalyst, if applicable, a mediator thereof; and
if applicable a substrate transporter.

39. The device according to claim 38, wherein each biosensor is able to measure an electrochemical signal and then comprises a working electrode, a reference electrode and a counter-electrode between which passes a current induced by the oxidation or reduction reaction of the substrate, the electrochemical signal being either the intensity of this current or the potential difference between the electrodes during the oxidation-reduction reaction of the substrate.

40. The device according to claim 39, wherein for each biosensor the working electrode is a carbon, gold or platinum electrode, the reference electrode is a platinum, gold or diamond electrode and the reference electrode is a silver chloride electrode.

41. The device according to claim 38, wherein the catalyst is an enzymatic catalyst or a chemical catalyst, it being possible for the chemical catalyst to be an abiotic catalyst chosen from platinum, platinum nanostructures, platinum alloy nanostructures, gold nanostructures and gold alloy nanostructures, or to be a molecular catalyst chosen from a porphyrin-gold complex and a porphyrin-rhodium complex.

42. The device according to claim 41, in which the catalyst is an enzymatic catalyst, wherein a mediator is associated with the enzymatic catalyst, the mediator being chosen from ferrocene, ferrocyanide, osmium complexes, quinone derivatives, and phenothiazine derivatives.

43. The device according to claim 38, wherein the biosensors of each group differ in at least one parameter selected from:
p1: the amount of catalyst;
p2: the Michaelis constant of the catalyst, in case the catalyst is an enzymatic catalyst or the saturation limit of the catalyst in case the catalsyt is a chemical catalyst;
p3: the amount of a mediator of the catalyst, if present, in case the catalyst is an enzymatic catalyst; or
p4: the Michaelis constant of a substrate transporter if present.

44. The device according to claim 43, the biosensors (BC) being noted:
BC₁₁ . . . BC₁ᵢ . . . BC₁ₙ . . .
BC₂₁ . . . BC₂ᵢ . . . BC₂ₙ . . .
BCⱼ₁ . . . BCⱼᵢ . . . BCⱼₙ . . .
BCₘ₁ . . . BCₘᵢ . . . BCₘₙ . . .
m and n each being an integer, m being the number of groups and n being the number of biosensors in each group, the biosensors BC₁₁ to BC₁ₙ belonging to group 1, the biosensors BCₘ₁ to BCₘₙ belonging to group m,
between each biosensor in a group, a parameter selected from p1 to p4 is varied; and between the biosensors of two different groups, another of these parameters p1 to p4 is varied.

45. The device according to claim 38, wherein:
the substrate is glucose;
the catalyst is an enzymatic catalyst selected from a glucose oxidase, a glucose dehydrogenase and a cellobiose dehydrogenase;
the enzymatic catalyst mediator, if present, is selected from ferrocene, ferrocyanide, osmium complexes, quinone derivatives, and phenothiazine derivatives;
the substrate transporter, if present, is a glucose transporter which is selected from GLUT1, GLUT2, GLUT3, GLUT4, GLUT6, GLUT8, GLUT10 and GLUT12.

46. The device according to claim 38, wherein:
the substrate is lactate;
the catalyst is an enzymatic catalyst selected from a lactate oxidase or a lactate dehydrogenase;
the enzymatic catalyst mediator, if present, is selected from ferrocene, ferrocyanide, osmium complexes, quinone derivatives, and phenothiazine derivatives;
the substrate transporter, if present, is a lactate transporter which is chosen from MCT1, MCT2, MCT3, MCT4.

47. The device according to claim 38, wherein the biosensors are arranged on a support, the working electrode, the reference electrode and the counter-electrode being screen-printed on the support.

48. The device according to claim 47, wherein the support on which the biosensors are arranged is chosen from glass plates, plastic plates, ceramic plates, nylon plates, silicon plates, polystyrene-based films, or polyester sheets.

49. The device according to claim 47, wherein the catalyst is deposited on the working electrode of each biosensor by encapsulation, grafting, absorption or trapping.

50. The device according to claim 49, wherein the catalyst is an enzymatic catalyst and is present on the surface of the working electrode of each biosensor by being applied on the surface of the working electrode inside a protective layer, the protective layer being of chitosan, Nafion, polypyrrole, or polyacrylic acid, or of a conductive polymer selected from polyaniline, polylactic acid, polydopamine and polyethylene glycol.

51. The device according to claim 50, wherein the enzymatic catalyst mediator is enclosed with the enzymatic catalyst within the protective layer.

52. The device according to claim 47, wherein a substrate transporter is present by being applied as a layer to the working electrode or optionally on the protective layer comprising the enzymatic catalyst, its mediator optionally being present, by depositing a layer of proteoliposomes enclosing the substrate transporter.

53. The device according to claim 47, wherein the support and the biosensors carried by the support are coated with a layer of chitosan, poly(2-hydroxyethyl methacrylate), poly(4-vinylpyridine-co-styrene) or alumina.

54. The device according to claim 47, wherein the device is arranged on the skin of a user or is implanted in the user in order to determine a region in which the actual concentration of the substrate in a medium is located, the substrate being glucose and the medium being blood.
